(19) United States
(12) Patent Application Publication
(10) Pub. No.: US 2025/0110108 A1
(43) Pub. Date: Apr. 3, 2025

(54) METHOD FOR SYNCHRONOUS AND FAST DETERMINATION OF TOTAL ARSENIC AND CONTENTS OF ARSENIC METABOLITES IN URINE

(71) Applicant: GUANGDONG UNIVERSITY OF TECHNOLOGY, Guangdong (CN)

(72) Inventors: Taicheng AN, Guangdong (CN); Yingjun WU, Guangdong (CN); Mengdie QI, Guangdong (CN); Guiying LI, Guangdong (CN)

(73) Assignee: GUANGDONG UNIVERSITY OF TECHNOLOGY, Guangdong (CN)

(21) Appl. No.: 18/841,398

(22) PCT Filed: Mar. 14, 2023
(86) PCT No.: PCT/CN2023/081473

§ 371 Date: Aug. 26, 2024

(30) Foreign Application Priority Data
Mar. 28, 2022 (CN) 202210312114.4

(51) Int. Cl. G01N 33/487; G01N 27/626; G01N 30/72; G01N 30/86

(52) U.S. Cl. CPC G01N 33/48714; G01N 27/626; G01N 30/72; G01N 30/8631

(57) ABSTRACT

A method for synchronous and fast determination of total arsenic and contents of arsenic metabolites in urine, which relates to the technical field of analysis of toxic and harmful organic pollutants, can be applied to analysis of urine samples of a population with occupational exposure to high-arsenic substances and provide metabolic stage data of arsenic in individuals of the exposed population. According to the method, ICP-MS is used for rapidly measuring the total arsenic of a human urine sample, and when the total arsenic of the urine samples exceeds a preset warning line, the ICP-MS is switched to HPLC-ICP-MS for separation and quantitatively analysis of five metabolites in urine, including arsenic betaine, trivalent inorganic arsenic, dimethylarsinic acid, monomethylarsonic acid and pentavalent inorganic arsenic. The specific steps includes pretreatment of a urinary total arsenic sample, pretreatment of a urinary arsenic speciation sample, on-line separation, preparation of quantitative standard curves and detection of actual samples.

### TECHNICAL FIELD

The present invention belongs to the technical field of analysis of toxic and harmful organic pollutants and particularly relates to a method for synchronous and fast determination of total arsenic and contents of arsenic metabolites in urine.

### BACKGROUND

Accidental exposure or long-term occupational exposure to arsenic will cause the accumulation of arsenic and poisoning phenomenon in the body. A toxic effect of the arsenic depends on its speciation. For example, the toxicity of inorganic arsenic is higher than that of organic arsenic, and the toxicity of low-valence oxidized arsenic (As^{III}) is higher than that of high-valence oxidized arsenic (As^{V}). When entering the human body, the inorganic arsenic is metabolized through the liver and finally metabolized into dimethylarsinic acid (DMA^{V}) through redox and methylation processes. Because these arsenic speciations are easily soluble in water and have a half-life of only a few hours in the human blood, most of the arsenic substances can be excreted with urine after metabolism in the body. Therefore, a urinary arsenic level is applied to monitoring recent arsenic exposure situations of individuals and also clinically applied to serve as a biomarker of arsenic exposure. At present, most of the hospitals carry out analysis of urinary total arsenic to determine whether a patient has excessive arsenic, and non-toxic or low-toxic arsenic speciations are usually involved. For example, arsenic betaine (AsB) derived from food is generally considered to be non-toxic or low-toxic, which accounts for a certain proportion of total arsenic in the urine of individuals who have eaten seafood recently, leading to deviation in clinical determination about whether the individuals have excessive arsenic. In addition, during treatment of individuals with exposure to arsenic, when most of the inorganic arsenic in some individuals may have been metabolized and converted into DMA^{V}, excessive medication may also increase unnecessary physical and economic burdens on the individuals. Therefore, mere analysis of the urinary total arsenic may not only overestimate an arsenic exposure level of the individuals and increase a medication dose for treatment, but also overestimate a pollution level in an arsenic-polluted area and cause unnecessary panic to a local government and the general public.

At present, inductively coupled plasma mass spectrometry (ICP-MS) is mostly used internationally to detect low-concentration metals, due to the advantages of a low detection limit, high sensitivity and capability of testing almost all of the metals. In China, an atomic fluorescence method is used based on the urinary arsenic and urinary arsenic speciation analysis standard issued at present. However, the method involves a redox reaction, a hydrogenation reaction, etc., which is not a method for directly analyzing arsenic substances in samples. Moreover, when the atomic fluorescence method is used to measure urine arsenic, the hydrogenation reaction can be carried out when the urine samples undergo complete digestion, and errors may be caused in the process. The ICP-MS method can be used for directly analyzing the element arsenic without complete digestion of urine, and results are not affected. Furthermore, high performance liquid chromatography-inductively coupled plasma mass spectrometry (HPLC-ICP-MS) can be used for directly analyzing arsenic substances of various speciations after chromatographic separation, and results are more intuitive and relatively more accurate. However, application of the method is limited by the phenomenon of co-elution of AsB and As^{III} in a chromatographic column. Therefore, through successful chromatographic separation of AsB and As^{III}, namely through simultaneous use of the ICP-MS to analyze the urinary total arsenic and the HPLC-ICP-MS to analyze urinary arsenic speciation metabolites, the analysis efficiency can be greatly improved.

Therefore, it is necessary to establish an ICP-MS method for measuring urinary total arsenic and an HPLC-ICP-MS method for measuring urinary arsenic speciations. The present invention can realize rapid screening of the urinary total arsenic of a population and further realize rapid separation and quantification of arsenic substances of five speciations, including AsB, As^{III}, MMA^{V}, DMA^{V} and As^{V}, in urine, to obtain more reliable in vivo metabolic situations of arsenic.

### SUMMARY

In order to overcome the disadvantages and shortcomings of the prior art, the purpose of the present invention is to provide a method for synchronous and fast determination of total arsenic and contents of arsenic metabolites in urine. The method can rapidly prepare samples to further analyze the contents of AsB, As^{III}, As^{V}, MMA^{V} and DMA^{V} in the urine after determining the total arsenic in the urine.

The purpose of the present invention is realized through the following technical solutions.

A method for synchronous and fast determination of total arsenic and contents of arsenic metabolites in urine includes the following operating steps:
step S1, diluting an arsenic standard substance with nitric acid to prepare arsenic ion standard solutions with gradient concentrations, performing detection by ICP-MS (inductively coupled plasma mass spectrometry), and with the concentration as x, signal intensity as y and a matrix solution as a blank, drawing a total arsenic standard curve, where the matrix solution is a solution with an HNO₃ concentration of 1% by volume; and using a urine matrix solution to prepare arsenic betaine (AsB) standard solutions, trivalent inorganic arsenic (As^{III}) standard solutions, dimethylarsinic acid (DMA^{V}) standard solutions, monomethylarsonic acid (MMA^{V}) standard solutions and pentavalent inorganic arsenic (As^{V}) standard solutions with gradient concentrations, respectively, performing detection by HPLC-ICP-MS (high performance liquid chromatography-inductively coupled plasma mass spectrometry), and with the concentration as xᵢ, chromatographic peak integral signal intensity as yᵢ and the urine matrix solution as a blank, drawing standard curves of urinary arsenic speciation metabolites, respectively, including an arsenic betaine standard curve, a trivalent inorganic arsenic standard curve, a dimethylarsinic acid standard curve, a monomethylarsonic acid standard curve and a pentavalent inorganic arsenic standard curve, where the urine matrix solution is obtained by mixing artificial urine and an (NH₄)₂HPO₄ solution with a concentration of 12.5-34.0 mM at a volume ratio of 1:(4-9) and adjusting a pH value to 7.8-11.0;
step S2, adding a urine sample to a polypropylene centrifuge tube, adding nitric acid, subjecting the urine sample to ultrasonic treatment in a water bath, and to filtration to obtain a urinary total arsenic sample to be measured; and meanwhile, adding a urine sample to a polypropylene centrifuge tube, diluting and evenly mixing the urine sample with a sample diluent to obtain a diluted urine sample, subjecting the diluted urine sample to filtration, and transferring the diluted urine sample to a polypropylene liquid phase sample vial to prepare a urinary arsenic speciation metabolite sample to be measured;
step S3, analyzing the urinary total arsenic sample to be measured obtained in step S2 by ICP-MS, and calculating the content of total arsenic in the urinary total arsenic sample to be measured according to the total arsenic standard curve obtained in step S1; and step S4, when the content of total arsenic in the urinary total arsenic sample to be measured exceeds a preset warning line, switching to an HPLC-ICP-MS analysis mode immediately, subjecting the urinary arsenic speciation metabolite sample to be measured obtained in step S2 to chromatographic separation by an anion exchange chromatography column to obtain arsenic speciation metabolites, then detecting the arsenic speciation metabolites by HPLC-ICP-MS analysis after the chromatographic separation, distinguishing speciation of arsenic metabolites by comparing chromatographic peak retention time with a standard substance, and according to chromatographic peak areas detected and the standard curves of urinary arsenic speciation metabolites obtained in step S1, calculating to obtain concentrations of the arsenic speciation metabolites in the urinary arsenic speciation metabolite sample to be measured.

In step S1, the nitric acid has a concentration of 1% to 2% by volume; and the pH value is adjusted with NaOH.

In step S1, the artificial urine is commercially available synthetic urine containing sodium chloride, magnesium chloride, calcium chloride, sodium sulfate, sodium citrate, sodium oxalate, sodium dihydrogen phosphate, potassium chloride, ammonium chloride, urea, etc., has a pH value of 5.7 and has low metal background.

In step S2, the total arsenic standard curve is: y=1470.9x−11.328, R²=0.9997, a linear range is 0.15-100 μg/L, where y is the signal intensity, and x is the concentration of arsenic; the arsenic betaine standard curve is: y₁=16680.46x₁+17.02, R²=0.9998, a linear range is 0.5-100 μg/L, y₁ is the chromatographic peak integral signal intensity of arsenic betaine, and x₁ is the concentration of arsenic betaine;
the trivalent inorganic arsenic standard curve is: y₂=6548.20x₂+22.00, R²=0.9997, a linear range is 0.5-100 μg/L, y₂ is the chromatographic peak integral signal intensity of trivalent inorganic arsenic, and x₂ is the concentration of trivalent inorganic arsenic;
the dimethylarsinic acid standard curve is: y₃=17903.03x₃+0.00, R²=0.9998, a linear range is 0.5-100 μg/L, y₃ is the chromatographic peak integral signal intensity of dimethylarsinic acid, and x₃ is the concentration of dimethylarsinic acid;
the monomethylarsonic acid standard curve is: y₄=17562.67x₄+64.13, R²=0.9998, a linear range is 0.5-100 μg/L, y₄ is the chromatographic peak integral signal intensity of monomethylarsonic acid, and x₄ is the concentration of monomethylarsonic acid; and
the pentavalent inorganic arsenic standard curve is: y₅=17413.33x₅+239.60, R²=0.9998, a linear range is 0.5-100 μg/L, y₅ is the chromatographic peak integral signal intensity of pentavalent inorganic arsenic, and x₅ is the concentration of pentavalent inorganic arsenic.

In step S2, the urine sample is a fresh human urine sample collected before use, or a human urine sample that is stored in a polypropylene cryopreservation tube at −80° C. to 4° C., taken out of a freezing environment, thawed at room temperature and then shaken and evenly mixed by a mixing machine for later use.

In step S2, the ultrasonic treatment in the water bath is performed at a temperature of 20-50° C. for 5-60 min; the sample diluent is an (NH₄)₂HPO₄ solution with a pH value of 7.8-11.0 and a concentration of 12.5-34.0 mM; and the pH value is adjusted with NaOH.

In step S3, an analysis mode of the ICP-MS is helium collision mode, and analysis conditions are as follows: As⁷⁵ is selected as an element to be measured, an internal standard solution is pumped in by a peristaltic pump when analyzing the content of total arsenic in the urinary sample to be measured, so as to make the urinary total arsenic sample evenly mixed with the internal standard solution in a three-way tube before analysis, and matrix drift of the urinary total arsenic sample is corrected based on a drift degree of the internal standard; and the internal standard is germanium (Ge) or iridium (Y).

In step S4, the anion exchange chromatography column is an anion exchange chromatography column equal to or less than 10 μm, a column temperature is 10-40° C., and a sample injection quantity is 10-100 μL; conditions for the chromatographic separation are as follows: a mobile phase A is ultra-pure water, a mobile phase B is a 10-30 mM (NH₄)₂HPO₄ solution with the pH value being adjusted to 7.8-11.0 with sodium hydroxide or ammonia water, and a flow rate is 0.8-2.0 mL/min; and a gradient elution procedure is as follows: 0-2 min. B: 0%-50%; 2-5 min, B: 50%-100%; 5-11 min, B: maintained at 100%; 11-12 min, B: 100%-0%.

In step S4, in the HPLC-ICP-MS analysis, As⁷⁵ is selected as an element to be tested, and Cl³⁵ is selected as an interference element.

In step S4, the exceeding the preset warning line means that the content of total arsenic in the urinary total arsenic sample to be measured exceeds 0.032 mg/L (with reference to WS/T 665-2019 Safety Guidance Value of Urinary Arsenic for Population); and the switching to the HPLC-ICP-MS analysis mode includes specific operations that a sample injector is changed from an atomizer to LC in instrument control software, an ICP-MS sample tube is pulled out from the atomizer in hardware and connected to an outlet end of the chromatography column, and a high performance liquid chromatography module is connected to an inductively coupled plasma mass spectrometer.

Compared with the prior art, the present invention has the following advantages and effects.

(1) The content of total arsenic in urine is analyzed by ICP-MS in the present invention, a pre-treatment process is simple and rapid, and an analysis method has high stability and accuracy.

(2) An HPLC-ICP-MS method is used for analyzing the urinary arsenic speciations in the present invention. Compared with a liquid chromatography-atomic fluorescence method, the HPLC-ICP-MS method can detect all speciations of arsenic substances obtained by chromatographic separation without reacting with potassium borohydride to generate gaseous arsenic compounds (when atomic fluorescence is used as a detector, target compounds need to be converted into a gaseous state, and some arsenic speciations cannot react to generate gaseous hydrides). Therefore, more speciations of arsenic compounds can be analyzed by the HPLC-ICP-MS.

(3) The method can rapidly quantify the concentration of total arsenic in urine and then further analyze the concentrations of different speciations of metabolites in urinary arsenic, so as to understand different metabolic stages of arsenic. An HPLC-ICP-MS instrument used has high sensitivity, and the method is simple and rapid and has accurate quantification results.

(4) The present invention can be applied to analysis of urine samples of a population with occupational exposure to arsenic so as to obtain various speciations and contents of toxic and harmful arsenic metabolites in the samples, obtain metabolic stages of the arsenic in the body, and provide a scientific basis for the exposure of environmental pollutants and the protection of individuals after arsenic intake.

### BRIEF DESCRIPTION OF DRAWINGS

FIG. 1 shows chromatographic separation peaks of AsB, As^{III}, DMA^{V}, MMA^{V} and As^{V}.

FIG. 2 shows quantitative analysis of urinary arsenic speciation in 100 cases of a high exposure group in Example 2 and comparison of adduct and total arsenic analysis results of five speciations of arsenic substances.

FIG. 3 shows arsenic speciation concentration levels of high, medium and low exposure (control) groups.

### DETAILED DESCRIPTION OF EMBODIMENTS

The present invention is further described below in combination with specific examples and accompanying drawings, but should not be construed as limitations of the present invention. Unless otherwise indicated, technical means used in the examples are conventional means well known to persons skilled in the art. Unless otherwise specified, reagents, methods and apparatuses used in the present invention are conventional reagents, methods and apparatuses in the technical field.

### Example 1

The present example provides a method for synchronously and rapidly analyzing contents of total arsenic and five arsenic metabolites in urine and use. The method includes the following steps.

Step S1. Standard solutions were specifically prepared as follows.

During analysis of the total arsenic in the urine, arsenic ion standard solutions with concentrations of 0, 0.5, 1, 2, 5, 10, 20, 50 and 100 μg/L were prepared with 1% (v/v) HNO₃ as a standard curve matrix, where an R2 value of a standard curve was greater than 0.999. A whole process blank was detected before sample analysis, and a content obtained after deducting the whole process blank was a sample content. Detection was performed by ICP-MS to draw a total arsenic standard curve: y=1470.9x−11.328, R²=0.9997, a linear range was 0.15-100 μg/L, y was the arsenic signal intensity, and x was the concentration.

A urine matrix solution was prepared by mixing artificial urine (commercially available synthetic urine containing sodium chloride, magnesium chloride, calcium chloride, sodium sulfate, sodium citrate, sodium oxalate, sodium dihydrogen phosphate, potassium chloride, ammonium chloride, urea, etc., with a pH value of 5.7 and low metal background) and 30 mM (NH₄)₂HPO₄ with a pH value of 8.0 at a volume ratio of 1:4, the urine matrix solution was used to prepare AsB, As^{III}, DMA^{V}, MMA^{V} and As^{V} standard solutions with concentrations of 0, 0.5, 1, 2, 5, 10, 20, 50 and 100 μg/L, respectively, detection was performed by HPLC-ICP-MS, and with the concentration as xᵢ, the chromatographic peak integral signal intensity as yᵢ and the urine matrix solution as a blank, standard curves of urinary arsenic speciation metabolites were drawn, respectively. Results were used in integral of peak area, the R2 value of the standard curves was greater than 0.999, a whole process blank was detected before sample analysis, and a content obtained after deducting the whole process blank was a sample content.

The standard curves of AsB. As^{III}, DMA^{V}, MMA^{V} and As^{V} are as follows:
AsB: y₁=16680.46x₁+17.02, R²=0.9998, a linear range was 0.5-100 μg/L, y₁ was the arsenic betaine chromatographic peak integral signal intensity, and x₁ was the arsenic betaine concentration;
As^{III}: y₂=6548.20x₂+22.00, R²=0.9997, a linear range was 0.5-100 μg/L, y₂ was the trivalent inorganic arsenic chromatographic peak integral signal intensity, and x₂ was the trivalent inorganic arsenic concentration;
DMA^{V}: y₃=17903.03x₃+0.00, R²=0.9998, a linear range was 0.5-100 μg/L, y₃ was the dimethylarsinic acid chromatographic peak integral signal intensity, and x₃ was the dimethylarsinic acid concentration;
MMA^{V}: y₄=17562.67x₄+64.13. R²=0.9998, a linear range was 0.5-100 μg/L, y₄ was the monomethylarsonic acid chromatographic peak integral signal intensity, and x₄ was the monomethylarsonic acid concentration; and
As^{V}: y₅=17413.33x₅+239.60, R²=0.9998, a linear range was 0.5-100 μg/L, y₅ was the pentavalent inorganic arsenic chromatographic peak integral signal intensity, and x₅ was the pentavalent inorganic arsenic concentration.

Step S2. Samples were specifically prepared as follows.

A urine sample used below was a fresh human urine sample collected before use, or a human urine sample that was stored in a polypropylene cryopreservation tube at −80° C. to 4° C., taken out of a freezing environment, thawed at room temperature and then shaken and evenly mixed by a mixing machine for later use.

(1) The urine sample was added to a centrifuge tube and evenly mixed, then 0.5 mL of the urine sample was added to a polypropylene centrifuge tube, 4.5 mL of 1.2% (v/v) HNO₃ was added, ultrasonic treatment was performed in a water bath at 45° C. for 1 hour, and filtration was performed with a 0.45 μm filter membrane to obtain a urinary total arsenic sample to be measured.

(2) Meanwhile, 200 μL of the untreated urine sample was added to a polypropylene centrifuge tube, 800 μL of a sample diluent (pH=8.0, 30 mM (NH₄)₂HPO₄, adjusted with NaOH) was added and evenly mixed, and filtration was performed with a 0.45 μm filter membrane to obtain a urinary arsenic speciation metabolite sample to be measured in a polypropylene liquid phase vial.

Step S3. ICP-MS analysis was performed under the following conditions.

The urinary total arsenic sample to be measured obtained in step S2 was analyzed by ICP-MS, and the content of total arsenic in the urinary total arsenic sample to be measured was calculated according to the total arsenic standard curve obtained in step S1.

Before the ICP-MS was turned on, a collision reaction cell was purged with helium at 5.0 mL/min for 30-60 min, a torch tube position, a lens and a resolution/mass axis were required to be tuned after turning on, and a performance report was required to satisfy instrument detection sensitivity, where a response of a tuning mass number 89 was greater than 6000, and a detection mode was a helium mode.

As⁷⁵ was selected as an element to be measured, and Y was used as an internal standard element. The urinary total arsenic sample to be measured and an internal standard were transported to a three-way tube by a peristaltic pump for mixing and analysis, and matrix drift of the element to be measured was corrected based on changes of the internal standard.

Step S4. HPLC-ICP-MS analysis was performed under the following conditions.

When the content of total arsenic in the urinary total arsenic sample to be measured exceeded a preset warning line, an HPLC-ICP-MS combined mode was switched immediately, chromatographic separation was performed on the urinary arsenic speciation metabolite sample to be measured obtained in step S2 by an anion exchange chromatography column to obtain arsenic speciation metabolites, then the arsenic speciation metabolites were detected by HPLC-ICP-MS analysis after the chromatographic separation, different speciations of arsenic metabolites were distinguished according to the comparison of chromatographic peak retention time with a standard substance, and according to chromatographic peak areas detected and the standard curves of urinary arsenic speciation metabolites obtained in step S1, concentrations of the arsenic speciation metabolites in the urinary arsenic speciation metabolite sample to be measured were calculated and obtained.

The switching to the HPLC-ICP-MS analysis mode specifically includes that a sample injector was changed from an atomizer to LC, an ICP-MS sample tube was pulled out from the atomizer and connected to an outlet end of the chromatography column, sample introduction was changed to LC, HPLC combined batch processing (using a preset EPA 6020 method of the United States) was opened after a module was updated, an acquisition mode was changed to TRA, As and Cl were selected as elements to be measured, an acquisition time was set to 12 min, and parameters were turned as follows: a rotation speed of the peristaltic pump was changed to 0.3 rps, the atomizer was changed to 0.85 L/min, a compensation gas was changed to 0.15 L/min, an He gas flow in the collision cell was changed to 4.0 mL/min, a response of a mass number 59 was greater than 2000 during tuning, and 156/140 was less than 1%.

Conditions for high performance liquid chromatography separation include: an anion exchange chromatography column: Hamilton PRP-X100 (250 mm×4.1 mm, 10 μm) or an equivalent column, and a protection column: PRP-X100 Grd. A mobile phase A was ultra-pure water, and a mobile phase B was a 25 mM (NH₄)₂HPO₄ solution with a pH value of 8.0-8.9; an injection quantity was 100 μL, and a flow rate was 1 mL/min; a column temperature was 20-30° C.; and a gradient elution procedure was as follows: 0-2 min, B: 0%-50%; 2-5 min, B: 50%-100%; 5-11 min, B: maintained at 100%; 11-12 min, B: 100%-0%.

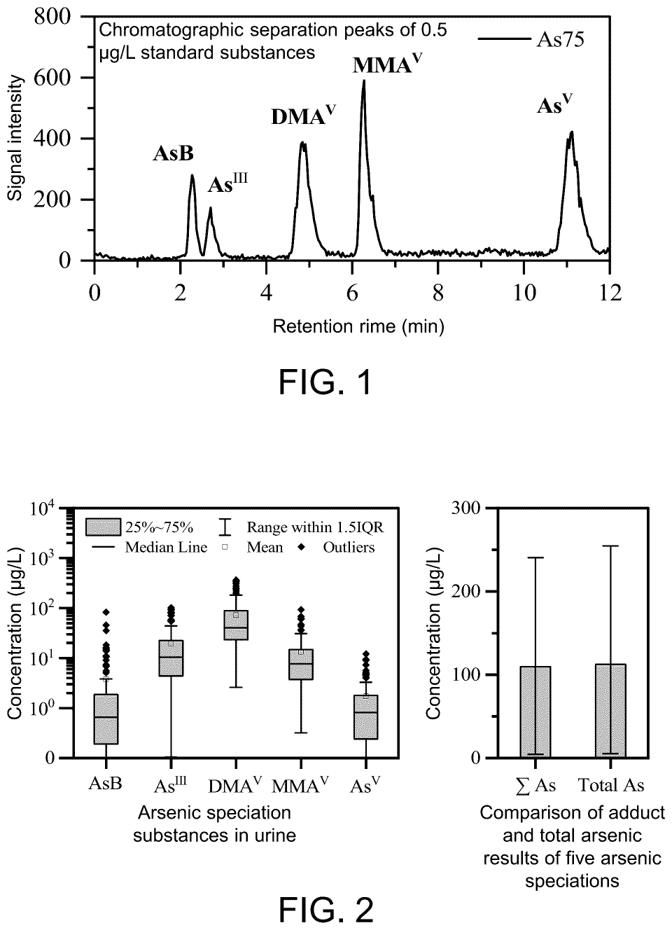

The chromatographic separation of five arsenic speciations in the present example is shown in FIG. 1, baseline separation of AsB, As^{III}, DMA^{V}, MMA^{V} and As^{V} can be realized within 12 min, and a detection limit is 0.1 μg/L. A detection limit of the analysis of total arsenic is 0.0425 μg/L.

### Example 2

In order to verify the accuracy and precision of the method provided in Example 1, spike-and-recovery and repeatability experiments were carried out using the method in the present example, and recovery rates of arsenite (As^{III}), monomethylarsonic acid (MMA^{V}) and dimethylarsinic acid (DMA^{V}) are verified by using a national standard substance GBW09115.

Testing method: At spiked levels of 0.5, 5 and 50 μg/L respectively, artificial urine was added to five arsenic speciation standard solutions at the low, medium and high levels, recovery rates and repeatability of arsenic speciations were determined by the detection method provided in Example 1, and the recovery rates of As^{III}, MMA^{V} and DMA^{V} in GBW09115 were verified. Results are shown in Table 1.

TABLE 1
Properties, retention time and recovery rates of arsenic speciation substances

| | | | | | | | Standard |
|---|---|---|---|---|---|---|---|
| | | | | | | | substance |
| | Abbre- | | Retention | Recovery | Intra-day | Inter-day | recovery |
| Name | viation | pka | rime (min) | rate | precision | precision | rate |
| Arsenic betaine | AsB | — | 2.272 | Low: | 4.6% | 6.7% | |
| | | | | 85.2% | 2.3% | 3.7% | |
| | | | | Medium: | 6.7% | 7.4% | |
| | | | | 74.1% | | | |
| | | | | High: | | | |
| | | | | 88.3% | | | |
| Arsenious acid | As^{III} | 9.2 | 2.639 | Low: | 5.1% | 4.4% | 86% ± 5% |
| | | | | 100% | 7.5% | 12% | |
| | | | | Medium: | 3.6% | 6.0% | |
| | | | | 104% | | | |
| | | | | High: | | | |
| | | | | 96.2% | | | |
| Dimethylarsinic | DMA^{V} | 6.2 | 4.543 | Low: | 6.0% | 5.1% | |
| acid | | | | 104% | 3.7% | 9.2% | |
| | | | | Medium: | 4.6% | 5.2% | |
| | | | | 96.9% | | | |
| | | | | High: | | | |
| | | | | 89.5% | | | |
| Monomethyl- | MMA^{V} | 4.6 | 6.012 | Low: | 1.4% | 5.6% | 86% ± 3% |
| arsonic acid | | | | 99.7% | 3.9% | 10% | |
| | | | | Medium: | 4.6% | 3.7% | |
| | | | | 96.0% | | | |
| | | | | High: | | | |
| | | | | 88.3% | | | |
| Arsenic acid | As^{V} | 2.3 | 10.922 | Low: | 4.0% | 7.3% | 82% ± 3% |
| | | | | 98.2% | 1.6% | 11% | |
| | | | | Medium: | 4.0% | 5.1% | |
| | | | | 99.6% | | | |
| | | | | High: | | | |
| | | | | 88.8% | | | |

Meanwhile, total arsenic standard solutions with concentrations of 2, 10 and 20 μg/L were added to blank urine, and a total arsenic recovery rate was measured by the detection method provided in Example 1. The recovery rate is 99.5% to 113%.

### Example 3

According to the method provided in Example 1, 100 cases of urine samples from individuals with occupational exposure to non-ferrous metal smelting were analyzed with a warning line preset as 0.032 mg/L. Results are shown in FIG. 2. Through comparison of the adduct (ΣAs) and total arsenic (tAs) of five arsenic speciation substances, mean values are 110±122 and 112±127 μg/L, respectively, indicating that the five arsenic speciations, including AsB, As^{III}, DMA, MMA and As^{V}, in the analyzed urine samples are main arsenic speciations present in the urine samples. Therefore, analysis of the five arsenic speciations, including AsB, As^{III}, DMA, MMA and As^{V}, can achieve the purpose of rapidly screening urinary arsenic metabolites of a population.

In addition, in the 100 cases of samples, 77 cases of samples have total arsenic exceeding the preset warning line, where AsB has a concentration range of 0-82.9 μg/L, an arithmetic mean value of 3.76±10.4 μg/L and a median of 0.817 μg/L, indicating that the samples have great differences in the content of AsB. The arsenic speciations that cause harm to the human body mainly include As^{III} and As^{V}, in the 77 cases of samples having the total arsenic exceeding the warning line, As^{III} has a concentration range of 2.13-101 μg/L and a median of 13.8 μg/L, and As^{V} has a concentration range of 0-12.1 μg/L and a median of 1.12 μg/L. As can be seen, in the 77 cases of urine samples, inorganic arsenic mainly has an occurrence speciation of As^{III}, which accounts for 3.8% to 36.9% of ΣAs and has highest toxicity in all arsenic speciations. Therefore, separation and quantification of As^{III} are extremely important and critical.

### Example 4

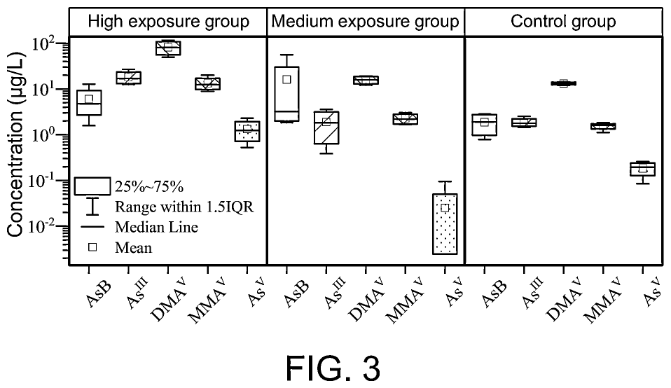

In order to verify that the method provided in Example 1 is applicable to different exposure population ranges, high, medium and low exposure groups (a population with occupational exposure to arsenic in metal smelting, a resident population around an exposure point and a control population, respectively) were set, 40 cases of urine samples were randomly selected from each group, and every 10 cases were mixed into 1 mixed sample for analysis. Results are as shown in FIG. 3. As can be found that in the three groups of samples, AsB accounts for a part of total arsenic, especially in the medium exposure group, indicating the separation of AsB and As^{III} has importance in urinary arsenic analysis. Secondly, in the three groups of samples, the five arsenic speciations were detected, indicating that the method is indeed applicable to a population with general exposure to arsenic.

The embodiments of the present invention are not limited by the above examples, and any other changes, modifications, substitutions, combinations and simplifications that are made without departing from the spiritual essence and principles of the present invention shall be regarded as equivalent replacement modes and are included in the scope of protection of the present invention.

1. A method for synchronous and fast determination of total arsenic and contents of arsenic metabolites in urine, comprising the following steps:
step S1, diluting an arsenic standard substance with nitric acid to prepare arsenic ion standard solutions with gradient concentrations, performing detection by ICP-MS, and with the concentration as x and signal intensity as y, drawing a total arsenic standard curve;
and using a urine matrix solution to prepare arsenic betaine standard solutions, trivalent inorganic arsenic standard solutions, dimethylarsinic acid standard solutions, monomethylarsonic acid standard solutions and pentavalent inorganic arsenic standard solutions with gradient concentrations, respectively, performing detection by HPLC-ICP-MS, and with the concentration as xᵢ, chromatographic peak integral signal intensity as yᵢ and the urine matrix solution as a blank, drawing standard curves of urinary arsenic speciation metabolites, respectively, comprising an arsenic betaine standard curve, a trivalent inorganic arsenic standard curve, a dimethylarsinic acid standard curve, a monomethylarsonic acid standard curve and a pentavalent inorganic arsenic standard curve, wherein the urine matrix solution is obtained by mixing artificial urine and an (NH₄)₂HPO₄ solution with a concentration of 12.5-34.0 mM at a volume ratio of 1:(4-9) and adjusting a pH value to 7.8-11.0;
step S2, adding a urine sample to a polypropylene centrifuge tube, adding nitric acid, subjecting the urine sample to ultrasonic treatment in a water bath, and to filtration to obtain a urinary total arsenic sample to be measured; and meanwhile, adding a urine sample to a polypropylene centrifuge tube, diluting and evenly mixing the urine sample with a sample diluent to obtain a diluted urine sample, subjecting the diluted urine sample to filtration, and transferring the diluted urine sample to a polypropylene liquid phase sample vial to prepare a urinary arsenic speciation metabolite sample to be measured;
step S3, analyzing the urinary total arsenic sample to be measured obtained in step S2 by ICP-MS, and calculating the content of total arsenic in the urinary total arsenic sample to be measured according to the total arsenic standard curve obtained in step S1; and
step S4, when the content of total arsenic in the urinary total arsenic sample to be measured exceeds a preset warning line, switching to an HPLC-ICP-MS analysis mode immediately, subjecting the urinary arsenic speciation metabolite sample to be measured obtained in step S2 to chromatographic separation by an anion exchange chromatography column to obtain arsenic speciation metabolites, then detecting the arsenic speciation metabolites by HPLC-ICP-MS analysis after the chromatographic separation, distinguishing speciation of arsenic metabolites by comparing chromatographic peak retention time with those of respective standard solutions in step S1, and according to chromatographic peak areas detected and the standard curves of urinary arsenic speciation metabolites obtained in step S1, calculating to obtain concentrations of the arsenic speciation metabolites in the urinary arsenic speciation metabolite sample to be measured.

2. The method for synchronous and fast determination of total arsenic and contents of arsenic metabolites in urine according to claim 1, wherein in step S1, the nitric acid has a concentration of 1% to 2% by volume; the pH value is adjusted with NaOH; and the artificial urine is commercially available synthetic urine.

3. The method for synchronous and fast determination of total arsenic and contents of arsenic metabolites in urine according to claim 1, wherein in step S1, the total arsenic standard curve is: y=1470.9x−11.328, R²=0.9997, a linear range is 0.15-100 μg/L, y is the signal intensity of arsenic, and x is the concentration of arsenic;
the arsenic betaine standard curve is: y₁=16680.46x₁+17.02, R²=0.9998, a linear range is 0.5-100 μg/L, y₁ is the chromatographic peak integral signal intensity of arsenic betaine, and x₁ is the concentration of arsenic betaine;
the trivalent inorganic arsenic standard curve is: y₂=6548.20x₂+22.00, R²=0.9997, a linear range is 0.5-100 μg/L, y₂ is the chromatographic peak integral signal intensity of trivalent inorganic arsenic, and x₂ is the concentration of trivalent inorganic arsenic;
the dimethylarsinic acid standard curve is: y₃=17903.03x₃+0.00, R²=0.9998, a linear range is 0.5-100 μg/L, y₃ is the chromatographic peak integral signal intensity of dimethylarsinic acid, and x₃ is the concentration of dimethylarsinic acid;
the monomethylarsonic acid standard curve is: y₄=17562.67x₄+64.13, R²=0.9998, a linear range is 0.5-100 g/L, y₄ is the chromatographic peak integral signal intensity of monomethylarsonic acid, and x₄ is the concentration of monomethylarsonic acid; and
the pentavalent inorganic arsenic standard curve is: y₅=17413.33x₅+239.60, R²=0.9998, a linear range is 0.5-100 μg/L, y₅ is the chromatographic peak integral signal intensity of pentavalent inorganic arsenic, and x₅ is the concentration of pentavalent inorganic arsenic.

4. The method for synchronous and fast determination of total arsenic and contents of arsenic metabolites in urine according to claim 1, wherein in step S2, the urine sample is a fresh human urine sample collected before use, or a human urine sample that is stored in a polypropylene cryopreservation tube at −80° C. to 4° C., taken out of a freezing environment, thawed at room temperature and then shaken and evenly mixed by a mixing machine for later use.

5. The method for synchronous and fast determination of total arsenic and contents of arsenic metabolites in urine according to claim 1, wherein in step S2, the ultrasonic treatment in the water bath is performed at a temperature of 20-50° C. for 5-60 min; the sample diluent is an (NH₄)₂HPO₄ solution with a pH value of 7.8-11.0 and a concentration of 12.5-34.0 mM; and the pH value is adjusted with NaOH.

6. The method for synchronous and fast determination of total arsenic and contents of arsenic metabolites in urine according to claim 1, wherein in step S3, an analysis mode of the ICP-MS is helium collision mode, and analysis conditions are as follows: As⁷⁵ is selected as an element to be measured, an internal standard solution is sucked in by a peristaltic pump when analyzing the content of total arsenic in the urinary total arsenic sample to be measured, so as to make the urinary total arsenic sample evenly mixed with the internal standard solution in a three-way tube before analysis, and matrix drift of the urinary total arsenic sample is corrected based on a drift degree of the internal standard; and the internal standard is germanium or iridium.

7. The method for synchronous and fast determination of total arsenic and contents of arsenic metabolites in urine according to claim 1, wherein in step S4, the anion exchange chromatography column is an anion exchange chromatography column equal to or less than 10 μm, a column temperature is 10-40° C., and a sample injection quantity is 10-100 μL; conditions for the chromatographic separation are as follows: a mobile phase A is ultra-pure water, a mobile phase B is a 10-30 mM (NH₄)₂HPO₄ solution with the pH value being adjusted to 7.8-11.0 with sodium hydroxide or ammonia water, and a flow rate is 0.8-2.0 mL/min; and a gradient elution procedure is as follows: 0-2 min, B: 0%-50%; 2-5 min, B: 50%-100%; 5-11 min, B: maintained at 100%; 11-12 min, B: 100%-0%.

8. The method for synchronous and fast determination of total arsenic and contents of arsenic metabolites in urine according to claim 1, wherein in step S4, in the HPLC-ICP-MS analysis, As⁷⁵ is selected as an element to be tested, and Cl³⁵ is selected as an interference element.

9. The method for synchronous and fast determination of total arsenic and contents of arsenic metabolites in urine according to claim 1, wherein in step S4, the exceeding the preset warning line means that the content of total arsenic in the urinary total arsenic sample to be measured exceeds 0.032 mg/L; and the switching to the HPLC-ICP-MS analysis mode comprises specific operations that a sample injector is changed from an atomizer to LC in instrument control software, an ICP-MS sample tube is pulled out from the atomizer in hardware and connected to an outlet end of the chromatography column, and a high performance liquid chromatography module is connected to an inductively coupled plasma mass spectrometer.
